(19) United States
(12) Patent Application Publication
(10) Pub. No.: US 2025/0352485 A1
(43) Pub. Date: Nov. 20, 2025

(54) PRODUCT COMPRISING A FORM RETAINING GRANULAR COMPOSITION OF GEL PARTICLES, A METHOD OF MANUFACTURING THE SAME AND A PROCESS OF FORMING A GRANULAR SLURRY

(71) Applicant: IAMFLUIDICS HOLDING B.V., Enschede (NL)

(72) Inventors: Tom Kamperman, Enschede (NL); Vasileios Dimitrios Trikalitis, Enschede (NL); Claas Willem Visser, Enschede (NL); Jeroen Rouwkema, Enschede (NL)

(21) Appl. No.: 18/873,428

(22) PCT Filed: Jun. 12, 2023
(86) PCT No.: PCT/IB2023/056034

§ 371 Date: Dec. 10, 2024

(30) Foreign Application Priority Data
Jun. 10, 2022 (NL) 2032118

(51) Int. Cl. A61K 9/50; A61K 8/11; A61Q 19/00

(52) U.S. Cl. CPC A61K 9/5036; A61K 8/11; A61K 9/5015; A61Q 19/00

(57) ABSTRACT

A product comprises a granular composition of gel particles, particularly micro-gel particles. Said composition comprises a carbohydrate matrix (lattice/network) between cells that contain said gel particles. A method of manufacturing such product that comprises a granular composition of gel particles, particularly micro-gel particles, involves forming an aqueous slurry comprising said gel particles and a water soluble carbohydrate compound. Said slurry is shaped into said product and said shaped slurry is solidified.

Product comprising a form retaining granular composition of gel particles, a method of manufacturing the same and a process of forming a granular slurry.

The present invention relates to a product, comprising a form-retaining granular composition of gel particles, particularly micro-gel particles. The invention further relates to a method of manufacturing a solid product of such kind and to a process of forming a granular slurry comprising gel particles. In this respect it is noted that the invention particularly relates to gel particles that are surrounded and confined by a hydrophilic polymer network. In this respect gel refers to a hydrogel composition which comprises a (hydrophilic) polymer network that can contain a significant amount of water such as in excess of 50 wt % and typically even more than 90 wt % of water.

Gel particles are found in myriad of products and markets, including food, life sciences and cosmetics. Forming products and devices that include such gel particles usually involves the use of a binder agent or the dispersion of them in a continuous medium. Known micro-gel particles include micro-capsules that comprise a fluidic core surrounded by a shell made out of a hydrogel. These hydrogel gel particles are produced in aqueous conditions and contain a relatively high concentration of an aqueous medium, which may add up to over 90%. This leads to a challenge of shelf life since the activation and degradation of the gel particles and/or their contents generally occurs in aqueous environments. Therefore, there is a need for preservation of the gel particle compositions and their ingredients.

The preservation of gel particles is subject to a wet state or a dry state. For wet state preservation, one or more additives may be added, which block or at least retards the reactivity of the particles and their contents. Examples of such preserving additives are for instance ethanol, salt solutions and parabens. The addition of these compounds, however, may cause unintended side effects in terms of health, environment and might also compromise the properties of the gel particles. Furthermore, wet state preservation usually results in liquid emulsions or soft particle aggregate form, limiting mechanical robustness, limiting the ability to supply predefined numbers of material in one single step, and preventing the ability to present the particle aggregate in a well-defined macroscopic shape.

In case of dry state preservation, substantially all water content from the gel particle composition may be removed. Examples of this process are solvent evaporation, and freeze-drying. Removing the water content from the gel particle composition, without affecting the original shape or properties of the gel particles, proves to be challenging. The formation of water crystals during freezing, or the stress that is exerted from the sublimation process onto the gel particles during solvent evaporation often proves sufficient to deform them and/or to compromise the macro-structure, their integrity and their contents. Accordingly the gel particle material needs to be optimized to endure the preservation process, which creates limitations to the materials that may be used for the gel particles. Particularly, the gel particles should be able to withstand freezing of their liquid content, and the removal of any liquid content during the drying step, in order to achieve dry state preservation.

To render the gel particles preservable in a dry state, it is known to modify the particle composition during production. For example, cross-linkable moieties may be included in the chemical composition that preserve irreversibly the shape of the gel particles. This, however, requires an additional step such as treatment of the particles with hazardous crosslinking chemicals like glutaraldehyde or formaline, or light-induced crosslinking of acrylate moieties via radiation with ultraviolet (UV) light. Exposing the composition and UV light may moreover damage the components within the gel particles. Furthermore, UV exposure is an in situ process, which is either necessary to be performed individually during production or in a relatively small composition volume which is limited by the maximum light penetration depth. Moreover this process is irreversible and results in a stiffer composition that may be incompatible with the targeted application.

Chemical cross linking may be used to induce preservation by introducing covalent cross-linking functional groups in the chemical structure of the shell compound of the gel particles. Covalent cross-links are usually stronger than other type of bonds, such as ionic cross-linking, and can render a gel particle strong enough to withstand freeze-drying. However, introducing covalent crosslinking moieties, such us methacrylate (MA) or dialdehyde (DA) groups complexes the production process in terms of chemistry, and drives up the cost of production.

Another issue is the preservation of the macro structure, i.e. the shape, of a product that was cast into shape out of a granular micro-particle composition. The formation of such a product usually involves compression of dry particles to the point of irreversible agglomeration (standard tablet compression) or freeze-drying of the composition. Thereby the shape of the macrostructure is preserved but only part of the particle constituent properties is retained such as flavour, texture and/or an active ingredient. The shape of the particles is likely to become irreversibly changed during compression, for example by their collapse, rupture, or plastic deformation.

Moreover, such a preservation method may be performed only once and the structure readily collapses upon (re) hydration. The latter is widely used as a method for dissolving tablets. However, re-freezing or compressing a composition containing flexible gel particles upon (re) hydration will not yield the same result. With each cycle of (re) hydration the properties readily shift towards a solution that contains gel particles of random shape rather than their initial morphology upon production.

Sintering a composition, having a carbohydrate phase, can improve the shape retention of the individual micro-particles. Sintering, however, is an irreversible process and usually the particles integrate with the sintered phase. As a consequence of such a process, the product loses its ability to unjam and the system can no longer return to a granular suspension state.

The present invention has inter alia as one of its objects to provide a product comprising a granular composition containing, and particularly substantially composed of, gel particles that meets the above limitations in that it allows for multiple cycles of preservation and (re) hydration while substantially retaining its shape and constituent properties.

In order to achieve said object, a product of the type as described in the opening paragraph, according to the invention, is characterized in that said composition comprises a solid structure of said gel particles bonded to one another by a substantially dry or dried carbohydrate matrix in between said gel particles.

The invention accordingly provides a carbohydrate interstitial matrix that provides a bonding structure in between the gel particles that are thereby held together. The carbohydrate layer, hence, gives the final product form-retaining consistency and structure. The carbohydrate matrix that is responsible for this structural cohesion, provides both this structural support to the product as well as an interstitial protective shield for the individual gel particles that are captured within the structure. Particularly, the total water (moisture) content of a dried form retaining product is less than 50 wt %, more particularly less than 15 wt %, even more particularly less than 10 wt % and even more particularly less than 5 wt %.

Particularly the product preserves the shape and size of the gel particles while also retaining its macroscopic shape, particularly during freeze-drying as well as upon (re) hydration. In its dry state condition, the product can be easily manipulated, such as being transported, packaged, administered or cut into smaller pieces, since it behaves as a solid material. But it also allows for multiple cycles of freeze-drying without any loss of properties on two scales. Particularly, the invention enables a long term storage and transport under ambient conditions of tablets consisting of materials that previously could not be freeze-dried without harming the properties and/or integrity of the product, notably of the gel-particles captured therein.

At this instance it is noted that the expression hydration or re-hydration, as used in this application, may refer to both aqueous solutions as well as to non-aqueous solutions or liquids. Also these expressions may inter-changeably be used with expressions like activation and re-activation or wetting and re-wetting.

In an further aspect of the invention, a method of manufacturing a solid product that comprises a granular composition of gel particles, particularly micro-gel particles, is characterized in that an aqueous slurry is formed comprising said gel particles and a water soluble carbohydrate compound, wherein said slurry is shaped, particularly moulded, and more particularly compressed, into said product, and wherein said shaped slurry, comprising said gel particles and a water soluble carbohydrate compound, is dehydrated and solidified to form said product.

In a particular embodiment, said method according to the invention is characterized in that said shape is solidified by at least substantially removing an aqueous content of said shape, particularly by drying, more particularly by freeze-drying. Particularly, the total water (moisture) content of the solidified and dried product is less than 50 wt %, more particularly less than 15 wt %, even more particularly less than 10 wt % and even more particularly less than 5 wt %.

Although many techniques may be used to shape said slurry into said product, a further particular embodiment of the method according to the invention is characterized in that said slurry is shaped by moulding, additive material printing or extrusion prior to solidification.

The original slurry may be regained again substantially unaltered by using a liquid activator that dissolves or disperses the carbohydrate lattice structure. To that end a process of forming a slurry that comprises a granular composition of gel particles, particularly micro gel particles, according to the invention, is characterized in that a product according to the invention is provided and said product is subjected to a liquid activator.

A preferred embodiment of the product according to the invention is characterized in that said carbohydrate matrix is water soluble. By being water soluble the carbohydrate matrix allows to be broken down simply by (re) hydration with an aqueous liquid activator. By using plain water or an aqueous solution as an hydration agent, a slurry is obtained that is compatible with many industrial applications, including applications in cosmetic industry, food industry, nutraceutical industry, or pharmaceutical industry such as edible compounds as used for oral drug delivery or ingestion of actives such as vitamins or pro/antibiotics.

A specific embodiment of the product according to the invention is therefore characterized in that said carbohydrate matrix comprises an amorphous matrix of at least one polycarbohydrate or polysaccharide compound, and more particularly in that said polycarbohydrate or polysaccharide compound comprises at least one sugar compound, and more particularly in that said sugar compound comprises dextran, dextrin, maltodextrin, trehalose, lactose, glucose, dextrose, sucrose, fructose, maltose, isomaltose, sorbitol, mannitol, lactitol, xylitol, and/or erythritol.

Accordingly, a preferred embodiment of the process of forming a slurry according to the invention, is characterized in that said aqueous liquid activator comprises an aqueous carbohydrate solution, particularly a poly-carbohydrate or polysaccharide solution, more particularly a sugar solution, even more particular an aqueous solution of dextran, dextrin, maltodextrin, trehalose, lactose, glucose, dextrose, sucrose, fructose, maltose, isomaltose, sorbitol, mannitol, lactitol, xylitol, and/or erythritol. Such a carbohydrate solution may reinstall a reinforming matrix within the composition upon a next solidification of the slurry, for instance by drying, particularly freeze-drying. This may allow for multiple (re) hydration cycles, over an over again, while at least substantially retaining the initial properties of the slurry composition, particularly of the gel particles contained therein.

A further specific embodiment of the product according to the invention is characterized in that said gel particles comprise a hydrogel, more specifically a hydrophilic polymer network, and more particularly in that said hydrogel comprises one or more polysaccharides selected from agar, alginate, chitosan, dextran, poly(ethylene glycol), collagen, gelatine, hyaluronic acid, carrageenan, fibroin, fibronectin, poly-l-lysine (PLL), cellulose, graphene, polyethylenimine (PEI), poly(amidoamine) (PAA), dextran sulfate, silk, silk fibroin, pectin, K-carrageenan, Iota carrageenan, gellan gum, guar gum, tragacanth gum, xanthan gum, acacia gum, karaya gum, locust bean gum, or sodium carboxymethyl cellulose (S-CMC). All of these materials are preferably applied as naturally derived materials and/or synthetically derived materials including recombinant proteins and/or derivatives of these materials, wherein said polymer network particularly comprises a calcium-alginate network. Particularly successful results were obtained in this respect with a further specific embodiment of the method and capsule, wherein said gel particles comprises a cross-linked or inter-penetrating alginate network, particularly a calcium cross-linked alginate network.

A further specific embodiment of the product according to the invention is characterized in that said gel particles comprise micro capsules having a core that is surrounded by a hydrophilic polymer network. Said micro-capsules may have a shell comprising both calcium alginate of a first molecular weight as well as calcium alginate of a second molecular weight, said second molecular weight being larger than said first molecular weight.

Said core may comprise at least one active, pharmaceutically, cosmetically, biologically active, or activatable, material from a group comprising; biologicals, anti-oxidants, vitamins, hormones, vaccines, microbiotics, probiotics, prebiotics, nucleic acids, antibiotics, enzymes, proteins, fungi, yeast, bacteria, plant cells, mammalian cells and stem cells, or other active compounds that are preferably shielded from the ambient in order to preserve them, that is, to prevent or reduce their activation and increase their shelf life or expiration date.

In a particular embodiment, the micro-capsules may have a fluid core. Said fluid core may comprise a mixture of immiscible liquids such as a water-in-oil emulsion or an oil-in-water emulsion. Said fluid core may comprise at least one liquid that is predominantly immiscible with water, especially an oil, especially liquid from a group comprising ethereal, macerated and/or essential oils or waxes furthermore adding favorable, pleasant organoleptic properties to the product. Examples of suitable organic lipophilic compounds are for instance:
vegetable oils, such as sunflower oil, corn oil, castor oil, palm oil, coconut oil, avocado oil, sweet almond oil, calophylum oil, lanolin, sesame oil, olive oil, jojoba oil, soybean oil, cottonseed oil, rapeseed oil, peanut oil, flaxseed oil, borage oil and vegetable oil derivatives,
essential oils, such as immortelle, lavender, german chamomile, neroli, peppermint oil, rosemary, rose oil, tea tree oil, dwarf pine, juniperberry, roast chestnut extract, birch leaf extract, hayseed extract, ethyl acetate, camphor, menthol, rosemary extract, eucalyptus oil,
macerated oils,
fatty acids, such as stearic acid, palmitic acid, behenic acid, myristic acid, lauric acid and capric acid,
fatty acid derivatives, such as fatty acid esters with short chain alcohols, such as isopropyl myristate, isopropyl palmitate and isopropyl stearate and dibutyl adipate; medium and long chain fatty acids and their esters with polyols such as propylene glycol);
animal oils, such as tallow fat and marine oils, such as fish oils and seaweed oils or mixtures thereof;
nut oils, seed oils, waxes, such as paraffin wax, carnauba wax, candelilla wax, beeswax, microcrystalline wax, ozocerite wax; and
triglyceride.

In a further specific embodiment, the micro-capsules have a core that is at least partly solid. Said completely solid or partially solid core may comprise a mixture of hydrophobic and hydrophilic materials. In a specific embodiment said hydrophilic material is distributed non-homogeneously through said hydrophobic core, and more specifically said mixture comprises pockets of said hydrophilic material within said hydrophobic material or said mixture comprises pockets of said hydrophobic material within said hydrophilic material.

In a further specific embodiment, said at least partly solid core comprises a degradable, particularly biodegradable material, specifically a polymer selected from a group, comprising: poly(lactic acid) (PLA), poly(glycolic acid) (PGA), poly(epsilon-caprolactone) (PCL), poly(lactic- co-glycolic acid) (PLGA), tri-methylene-carbonate (TMC), as well as modifications of these materials, particularly block copolymers comprising poly(ethylene glycol) (PEG) as one of the blocks, more particularly PEG-PLA, PEG-PLGA, PEG-PGA, or PEG-PCL. Other materials are poly-ortho-esters (POE), particularly fourth generation POE (POE IV).

In a further specific embodiment such product according to the invention provides a sustained-release composition, releasing a bio-active agent. More specifically, said at least partially solid core may undergo degradation under physiological conditions and more specifically degrades partly or completely via hydrolysis. Said degradation may be a result of bulk erosion or surface erosion. By hydrolyses in an aqueous environment, the core will erode, thus freeing en releasing the active agent that was captured therein.

In a further specific embodiment said degradation results in the release of one or more active agents from the core and more specifically said degradation results in the delivery of said active agent to the surrounding. Said degradation and/or said delivery may happen in a timely controlled manner, specifically during the time course of at least one day, more specifically at least one week, more specifically at least more than one month.

The product according to the invention is characterized in that said product comprises gel micro-particles or micro-gels which are suitable for administration by intramuscular, intravenous, subcutaneous, intra-articular or intra-peritoneal injection. The product according to the invention is characterized in that said product is entirely or partly suitable for administration to the eye, nose, oral cavity, gastrointestinal tract or vaginal cavity.

A further specific embodiment of the product according to the invention is characterized in that said gel particles have a size in a range of between 1 micron and 5 millimetre, specifically in a range of between 1 micron and 500 micron. Particularly these particles have a coefficient of variation in size of less than 10%, preferably less than 5%. In case of non-spherical particles the above size refers to their Feret diameter.

A further specific embodiment of the product according to the invention is characterized in that at least part of said gel-particles are capable of interacting with biological cells, particularly by being functionalized with one or more compounds selected from a group, comprising: nucleic acids (aptamers), proteins and peptides.

Said gel particles may be biological cell carriers or cell-adhesive micro-particles. These particles may contain a positively charged surface for example resulting from the presence of a polyelectrolyte such as poly-l-lysine. Alternatively the micro-particles may contain a cell-adhesive (natural) polymer or a protein such as gelatin, collagen, fibronectin or laminin, or alternatively a polymer functionalized with cell-adhesive moieties such as cellular integrin binding peptide sequences containing arginine-glycine-aspartic acid (RGD) or cellular cadherin binding peptide sequences containing histidine-alanine-valine acid (HAV), or combinations of all of the above

Gel particles with a size between 10 and 100 micron are particularly suitable for pharmaceutical applications. For the purpose of food and nutrition gel particles having a size between 100 and 500 micron may be used. In cosmetics, personal care and agriculture the gel particles will generally have a size between 500 and 5000 micron. However, it will be appreciated that in any of these fields of application the gel particle size may also be chosen outside those ranges to suit a specific application in particular.

A further specific embodiment of the product according to the invention is characterized in that said composition comprises at least 50% of volume of said gel particles and at least 10% of weight of said carbohydrate matrix. The volume fraction of said particles is defined as the total volume of the gel particles, when swollen in water, in the granular material relative to a total volume of the respective granular material.

A volume fraction beyond 50% is especially selected to approach or even exceed that of a random close packing (approx. 64% v/v) and even more especially approaching or even exceeding the maximum packing of non-deformable spheres (approx. 74% v/v). The volume fraction may especially be in the range of 50-95% v/v, such as in the range of 60-90% v/v, particularly I a range of 75-90% v/v. Further, the particles may be elastically deformable. In order to achieve a volume fraction of over 74%, the particles may especially be selected for being deformable. The volume fraction relates to (physical) properties of the particles. For instance, the range of volume fractions in which said particles may form a form-retaining, i.e shape-stable, product according to the invention may be different for a granular material comprising stiff particles than for another granular material comprising weaker particles.

A further specific embodiment of the product according to the invention is characterized in that said composition is shaped to form at least a portion of a tablet, particularly a tablet that is at least ten times a volume of an average volume of said gel particles. Such tablet may have structural gradients and compartments resulting in further patterning of the additives over time.

Particularly said tablet may comprises multiple compartments and said composition is shaped into one of said compartments of said tablet. Further, the tablet may be textured or engraved on its surface and/or the tablet may be prepared to be cut or broken into distinct pieces, particularly by (partially) laser cutting the dry tablet. The tablet may exhibit a surface texture or gradients in the particle packing, local chemistry, or local physical properties to promote division of the tablet in distinct pieces after activation. Specifically, in a fashion that each piece contains a controlled amount of microgels.

In a further specific embodiment, the product according to the invention is characterized in that said shaped slurry is cast or moulded in, or coated by an elastomer compound prior or past solidification, particularly in or by polydimethylsiloxane (PDMS). Such elastomer mould or coating acts like a cartridge that captures the composition inside and has the ability to expand together with the product while it is being (re) hydrated. The product in said cartridge can be reactivated, particularly hydrated, by penetrating of the coating and introducing an (aqueous) liquid activator through the channel thus obtained. The elastomer structure may be sufficiently porous to allow air to flow out while the liquid enters the interior space.

In a further specific embodiment, the micro gel particle slurry may be cast, other wise shaped or injected into a silicone 3D mould. It is then frozen by submerging in liquid nitrogen, or via directional freezing by placing atop a cold surface. The sample can be dried in a freeze-dryer. After drying, the dried microgel-tablet is shape stable, i.e. form retaining, and can be retrieved from the mould. Activation by rehydration with a liquid can be done inside or outside the mould.

A further specific embodiment of the product according to the invention is characterized in that said composition moreover comprises an effervescent disintegration agent, particularly a carbonate compound. Such effervescent agent may boost the disintegration of the product upon contact with a suitable liquid, particularly water, while a gaseous compound, particularly oxygen or carbon dioxide is produced and escapes. Particular applications are as a food supplement, flavour, or reduced-fat product, and in cosmetics, like (water-free) bathing products or facial creme. Particular applications are also as a pharmaceutical construct containing an active pharmaceutical ingredient, or as a product for agriculture containing an active agrochemical compound such as a (biological) pesticide, or as a cosmetic product containing a microbiological compound, or a fragrance product containing a natural fragrance compound. Another particular application is as an edible product for oral delivery of an active compound such as an active pharmaceutical or nutraceutical ingredient like a probiotic strain.

A further specific embodiment of the product according to the invention is characterized in that said composition moreover comprises a plasticizer agent, particularly an ethanol. The granular composition can become malleable upon addition of a weakening agent such as ethanol.

The product may be a medicinal product, wherein said gel particles comprise a pharmaceutically active agent. These particles typically may have a size between 10 and 100 micron.

The product may be a beauty product, wherein said gel particles comprise a cosmetically active agent. Such particle typically may have a size between 500 and 5000 micron.

The product may be a food product, wherein said gel particles comprise a nutritious ingredient or supplement. The gel particle in such case may typically have a size between 100 and 500 micron.

Hereinafter, the invention will be described in further detail with reference to a specific embodiment and an accompanying drawing. In the drawing:

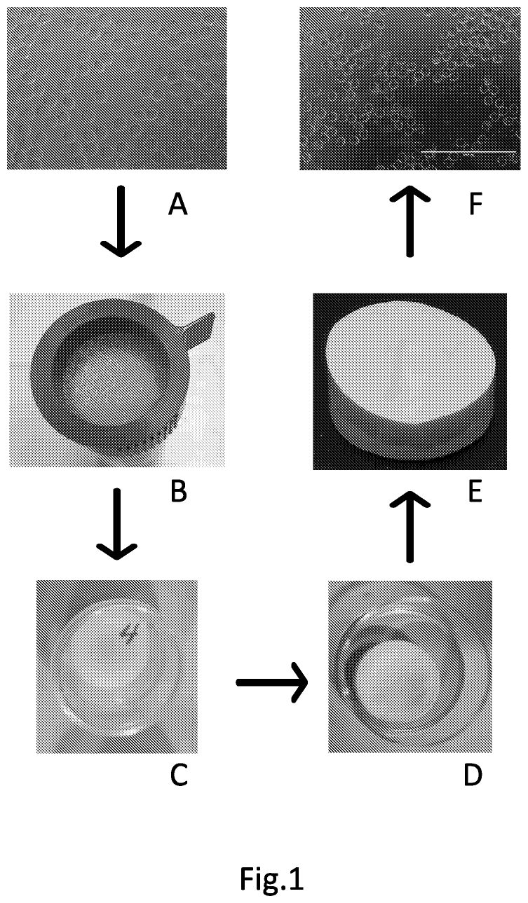

FIG. 1A-1F microscopic pictures of a device and composition according to the invention in subsequent stages of processing; and

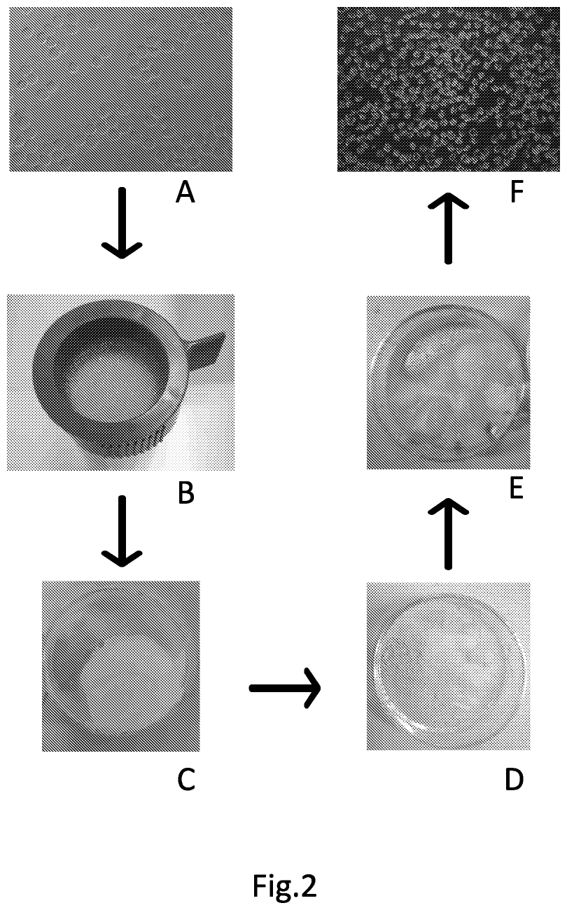

FIG. 2A-1F microscopic pictures of a shadow device and composition according to the prior art in subsequent stages of processing;

It is noted that some figures may be drawn purely schematically and not necessarily to a same scale. In particular, certain dimensions may have been exaggerated to a more or lesser extent to aid the clarity of any features. Similar parts are generally indicated by a same reference numeral throughout the figures.

Before the any products, compounds, compositions, formulations, devices, methods, or uses are disclosed and described in this application, it is to be understood that the aspects described below are not limited to specific products, compounds, compositions, formulations, devices, methods, or uses as such may, of course, vary. It is also to be understood that the terminology used herein is for the purpose of describing particular aspects only and is not intended to be limiting.

It must be noted that, as used in the specification and the appended claims, the singular forms “a,” “an” and “the” include plural referents unless the context clearly dictates otherwise. Thus, for example, reference to “an active agent” includes mixtures of two or more such agents, and the like.

The present invention relates to a method for preparing a product having a granular composition, the grains of which are being formed by gel particles, particularly gel micro-particles. The term “particles” as used herein is interchangeable with “spheres”, “beads”, “pearls” or “capsules” and, particularly refers to particles that comprise a core which is surrounded by a shielding and/or core confining polymer network of a hydroplilic gel compound or composition.

The gel particles may contain an active agent or other substance dispersed, dissolved or otherwise distributed therein. The gel particles are usually made up of substantially uniform particles of a spherical shape, although sometimes the gel particles may be irregularly shaped. The uniform gel particles can be in the size range (diameter) from submicron to millimetre. Particularly these particles have a coefficient of variation in size of less than 10%, preferably less than5%. In case of non-spherical particles the above size refers to their Feret diameter.

The term “active agent” as used herein is interchangeable with “bio-active agent”, “cosmetically active agent”, “pharmaceutically active agent”, or “drug” and refers to an agent which has biological activity and used to treat, diagnose, cure, mitigate, prevent (i.e., prophylactically), ameliorate, modulate, or have an otherwise favorable effect on a disease, disorder, infection, and the like. Active agents also include a pro-drug which becomes bioactive or more bioactive after it has been placed in a predetermined physiological environment.

Various forms of the active agent can be used, which are capable of being released from the gel particle into adjacent tissues or fluids. To that end, a liquid or solid active agent can be incorporated into the cores of the gel particles described herein. As such, the active agents can be acidic, basic, or amphoteric salts. In some embodiment, the active agents can be nonionic molecules, polar molecules, or molecular complexes capable of hydrogen bonding. The active agent can be included in the gel particles in the form of, for example, an uncharged molecule, a molecular complex, a salt, an ether, an ester, an amide, polymer drug conjugate, or other form to provide the effective biological or physiological activity.

Examples of a salt include, in the case that the active agent has a basic group such as an amino group, a salt with an inorganic acid (referred to also as an inorganic free acid) (e.g., carbonic acid, bicarbonic acid, hydrochloric acid, sulfuric acid, nitric acid, boric acid, etc.), an organic acid (referred to also as an organic free acid) (e.g., succinic acid, acetic acid, propionic acid, trifluoroacetic acid, etc.) or the like. Examples of the salt include, in the case that the active agent has an acidic group such as a carboxyl group, a salt with an inorganic base (referred to also as an inorganic free base) (e.g., alkaline metal such as sodium and potassium, alkaline earth metal such as calcium and magnesium, etc.), an organic base (referred to also as an organic free base) (e.g., organic amines such as triethylamine, basic amino acids such as arginine, etc.) or the like. Moreover, the physiologically active peptides may form a metal complex compound (e.g., a copper complex, a zinc complex, etc.).

Examples of active agents that can be incorporated into gel particles herein include, but are not limited to, biologicals, anti-oxidants, vitamins, hormones, vaccines, microbiotics, probiotics, prebiotics, antibiotics, enzymes, proteins, fungi, yeast, bacteria, plant cells, nucleic acids, mammalian cells and stem cells. In some embodiment, the active agent may be water-soluble or water-dispersible. In some embodiment, the active agent may be soluble or dispersible in a solvent such as organic solvent or inorganic solvent. The active agent may be dissolved or dispersed in an aqueous solvent. One non-limiting example of the aqueous solvent is water.

The “core” as used herein may comprise a bio-degradable and bio-compatible polymer or a lipid that captures, encapsulates, binds or otherwise contains an active agent that is to be released onsite. Suitable biodegradable polymers include, but are not limited to, poly(glycolic acid), poly(d,l-lactic acid), poly(I-lactic acid), copolymers of the foregoing including poly(d,l-lactide-co-glycolide) (PLGA), poly(aliphatic carboxylic acids), copolyoxalates, poly(caprolactone), poly(dioxanone), poly(ortho carbonates), poly(acetals), poly(lactic acid-caprolactone), polyorthoesters, poly(glycolic acid-caprolactone), polyanhydrides and polyphosphazines, or derivatives thereof, or combinations thereof. In some embodiment, the biodegradable polymer comprises block copolymers of hydrophilic and hydrophobic polymers.

The present invention provides a method and composition to increase the shelf life of soft micro-particles, particularly hydrogel particles, and capsule tablet compositions that does not require irreversibly annealing them at a post production time point, thus avoiding modifications of the micro-particle material. As a result the micro-particle composition may be re-hydrated and subsequently dried multiple cycles again. Alternatively, the micro-particles might be used or consumed upon first rehydration.

As an explanatory embodiment a hydrogel gel composition was prepared according to the invention. This composition is shown in consecutive stages of processing in FIGS. 1A-1F and comprises an aqueous solution of a water soluble carbohydrate compound. In this example a sugar compound was selected as said carbohydrate compound, namely maltodextrin. Spherical calcium-alginate micro-particles made from 0.25% w/v sodium alginate (Wako, 80-120 cP at 1%) in water cross-linked using 0.2M CaCl2) in water with an average diameter of about 100 micron were infused with said aqueous solution containing 60% w/v maltodextrin (MDX13-17) in water for 2 hours. FIG. 1A gives a microscopic image of the starting phase of this gel composition according to the invention.

For comparison similar alginate micro-particles were infused with demineralized water to render a control gel composition. This shadow composition comprises an aqueous gel of 0.25% (w/v) sodium alginate micro-particles cross-linked with calcium and infused with water. This shadow composition is shown in consecutive stages of processing in FIGS. 2A-2F. FIG. 2A gives a microscopic image of the starting phase of this gel composition that is provided for comparison.

Both starting gel compositions are being moulded into shape, as shown in FIGS. 1B and 2B respectively, to form a tablet. A cup shape mould was used in which the microgel slurry was received without exerting hardly any pressure on the slurry itself. Both tablets have a disk shape with a diameter of approximately 20 millimetre and a thickness of approximately 5 millimetre. The total volume of each tablet is more than a million times a volume of an average volume of said gel particles.

The cups with the microgel slurry are placed upside down atop a sieve or strainer with a mesh size smaller than the microgel particles. The slurry is concentrated by removing part of the aqueous solution, until a microgel/liquid volume fraction is reached that is high enough to yield a malleable, shape-stable, jam-packed slurry. For calcium-alginate micro-particles this is above a volume fraction of about 74%. The moulded product is then being solidified by submerging in liquid nitrogen as shown in FIGS. 1C and 2C, respectively. Besides such snap freezing by submerging in liquid nitrogen, the product may also be subjected to directional freezing atop a cold surface of less than minus 80 degrees Celsius.

Already in this frozen state the carbohydrate matrix provides consistency to the product of FIG. 1C that has kept its shape. The control product of 2C already lost part of its consistency. Upon drying this difference becomes even more prominent. As is clear from a comparison between FIGS. 1D and 2D, the carbohydrate compound has formed an carbohydrate amorphous matrix between the gel particles. This carbohydrate lattice and network has maintained the shape and consistency of the moulded product, i.e., the tablet that is shown in FIG. 1D. The shadow product, on the other hand, lacks such an carbohydrate structure and fully disintegrated into micro-particle powder.

The dried product is infused with liquid again, such as water, the re-hydrated shadow product (FIG. 2E) immediately forms a shapeless gel slurry, whereas the product containing the reinforcing carbohydrate matrix (FIG. 1E) still maintains its macroscopic shape and integrity as long as the micro-particle volume fraction is still above typically about 74%. The latter is also the case on a microscopic scale as shown in the microscopic images of FIGS. 1F and 2F respectively. Clearly the micro-particles remained substantially unaltered between the stages of FIGS. 1A and 1F after having undergone a complete freeze-drying and re-hydration cycle. The micro-particles in the control composition, on the other hand, that underwent the same processing steps are considerably damaged. If more liquid, such as water is added, to the product of FIG. 1E the macroscopic construct will finally disintegrate into a slurry or dilute suspension of individual micro-particles when their volume fraction drops below said 74%.

It has been shown that the product according to the present invention, as shown in FIGS. 1A-1F, could withstand multiple re-hydration and drying cycles over and over again without substantially affecting the macroscopic shape of the product nor the microscopic integrity of the soft calcium-alginate gel-particles contained therein.

Although the invention was elucidated in more detail with reference to merely a limited number of embodiments, it will be appreciated that the invention is by no means limited to those embodiments. To a skilled person, many other embodiments and variations are on the contrary feasible within the scope of the present invention without requiring any inventive skill or labour.

Similar results were achieved with shock freezing of a tablet with total volume that is at least 10 times more that the particle unit volume. It appeared possible to infuse, mould, freeze, dry and reconstitute a granular composition consisting of hydrogel particle units by rapid cooling in a freezer or on a freezing surface to minus 80 degrees Celsius or below.

Products of shapes other than a tablets may be created according to the invention by soft microscopic gel-particles. Even products having complex geometries remain preserved upon drying and re-hydration or activation due to the reinforcing carbohydrate lattice.

Similar results are achieved with micro gel-particles or micro gel-fibers of different size, shape and/or composition. Particularly half-spherical micro-particles of the order of 1 millimetre were able to maintain their semi-spherical shape upon freezing, drying and re-hydration. Particularly the micro-particles may be infused with a plasticizer like a 70% ethanol solution to improve the flexibility and deformability of the product.

Particularly the re-hydrated or otherwise re-activated product may deliver a slurry that is extrudable or jetable (3D printing) or injectable (pharmaceuticals) by the addition of a suitable plasticizer or other weakening agent. The product may be shaped out of micron scale gel-particles as a bar with a tailored cross-section that fits the micro channels of a microfluidic device that was etched or moulded into a material like glass, silicon or a polymer such as PDMS (PolyDimethylSiloxane). Likewise such product may be formed into a column that can be (pre-) loaded easily into a syringe for injectable purposes.

The micro-particle carbohydrate sub units may comprise all types of physically (e.g. ionically) and/or chemically (i.e., covalently) cross linked hydrogel beads including agar, alginate, chitosan, dextran, poly(ethylene glycol), collagen, gelatine, hyaluronic acid, carrageenan, fibroin, fibronectin, poly-l-lysine (PLL), cellulose, graphene, polyethylenimine (PEI), poly(amidoamine) (PAA), dextran sulfate, silk, silk fibroin, pectin, K-carrageenan, Iota carrageenan, gellan gum, guar gum, tragacanth gum, xanthan gum, acacia gum, karaya gum, locust bean gum, or sodium carboxymethyl cellulose (S-CMC). All of these are preferably applied as naturally derived materials and/or synthetically derived materials including recombinant proteins and/or derivatives of these materials, wherein said polymer network particularly comprises a calcium-alginate network. As an example, similar results were achieved with 5% (w/v) water-swollen gelatine particles of between 1 and 500 micron that were cross-linked using formaldehyde and infused with 60% maltodextrin (MDX 13-17; i.e. said aqueous solution).

The gel-particle sub units may constitute various morphologies, including solid matrix, core/shell capsules, multicore capsules, compartmentalized capsules, compartmentalized particles. The gel particle may contain an active agent that is released instantaneously or over time (time release) once the product is activated, for instance by being hydrolysed. The active agent, while being preserved in a dry state, may contain an active cosmetic, beauty agent, a nutritious additive, a nutritious supplement, a pharmaceutical active ingredient, an agrochemical ingredient or other ingredient like pigments or soy lecithin.

As an example, the method and capsule according to the invention may be used in embodiments, wherein said active compound comprises at least one vitamin, particularly a vitamin that is selected from a group containing thiamine, riboflavin, nicotinic acid, pantothenic acid, pyridoxine, biotin, folic acid, cyanocobalamin, lipoic acid, ascorbic acid, lecithin, glycyrrhizin acid, retinol, retinol palmitate, tocopherol, tocopherol acetate, salicylic acid, benzoyl peroxide, and azelaic acid and/or derivatives thereof, more particularly ascorbic acid and/or derivatives thereof.

As another example, the method and capsule according to the invention may be used in embodiments, wherein said active compound comprises at least one anti-oxidant, particularly an anti-oxidant that is selected from a group containing poly-phenols, thiol-based components, sulphite and derivatives thereof. These active compounds may be used, for instance, as nutritious supplements or for pharmaceutical treatment, in which case they are likely to be administered orally. The micro-particles and/or micro-particle-laden tablet may be formulated to survive the acidic environment of the human stomach to be digested in the more downstream portion of gastrointestinal tract of the user to release its contents. Specifically pro- and prebiotics may be administered particularly effectively in this manner.

Other applications of the micro-particles according to the invention may be in paint, carbon capture, fillers, building materials (concrete), smart materials that respond to stress and/or temperature.

Particularly the micro-particles may comprise micro gel-capsules that contain a bio-active compound like enzymes, cells, proteins and pharmaceutical agents. Mammalian cells, stem cells, yeast, plant seeds, fungi, bacteria or other living micro-organisms can be added within the liquid activator. The carbohydrate space between the hydro gel subunits patterns the architecture of proliferation of such living additives.

The composition or product can be used as an assembly part of a larger granular composition or product, particularly for creating a multi-component product like a tablet. And also other activators than water may be used for infusion and re-activation of the product and composition. By adding a surplus of activator finally the product may disintegrate. The disintegration time may be dependent on the macroscopic shape and size of the product as well as those of the constituent microscopic gel-particles.

A uniformity mass of the dried tablets may be aligned with the pharmacopeia standard for pharmaceutical applications. A total of 75 tablets of equal size were produced by freeze-drying mixtures of micro-particles and an interstitial carbohydrate matrix. The micro-particles are water-swollen microgels made of calcium cross-linked alginate (0.25 wt % alginate cross-linked using 0.2 M CaCl₂) in water). The interstitial carbohydrate matrix comprises a maltodextrin solution of 60% matodextrin in water. Of these particles, 22 were randomly chosen and weighed 3 times with a high accuracy weight scale. None of the tablets has more than a 10% deviation from the average mass as stated in the standard of pharmacopoeia for uniformity of mass regarding uncoated tablets.

The product can include an effervescent disintegration agent for carrying or releasing one or more active compounds in certain applications, including pharmaceutical effervescent tablets, food applications as in drinks or wellness or personal care applications like bathing. To that end for instance an acid or carbonate compound may be added. The hydrogel particles may function as an active ingredient (e.g. pharmaceutical, food or beauty) carrier with a controlled, passive release profile upon activation tailored to the application, while preserving the shelf-life of their properties.

A functional device, such as a sensor and/or an actuator may be embedded within or on the surface of the composition prior to freezing or after drying. The product may be embedded or otherwise encapsulated with an elastomer matrix, like PDMS, to provide a flexible casing. The product can be re-hydrated within the PDMS casing.

The surface gradients of the product may dictate a flow of the liquid added after drying. Also the carbohydrate network of the composition or gradients in the particle properties within the dried product may dictate the flow of the liquid added after drying.

Particularly the carbohydrate network of the composition may control the capillary flow route of the liquid added after drying.

1. A product comprising a form-retaining granular composition of gel particles, particularly micro-gel particles, wherein said composition comprises a solid structure of said gel particles bonded to one another by a substantially dry or dried carbohydrate matrix in between said gel particles.

2. The product according to claim 1, wherein said carbohydrate matrix is water soluble.

3. The product according to claim 1, wherein said carbohydrate matrix comprises an amorphous matrix of at least one polycarbohydrate or polysaccharide compound, and more particularly wherein said polycarbohydrate or polysaccharide compound comprises at least one sugar compound.

4. The product according to claim 3, wherein said sugar compound comprises dextran, dextrin, maltodextrin, trehalose, lactose, glucose, dextrose, sucrose, fructose, maltose, isomaltose, sorbitol, mannitol, lactitol, xylitol, and/or erythritol.

5. The product according to claim 1, wherein said gel particles comprise a hydrogel, more specifically a hydrophilic polymer network.

6. The product according to claim 5, wherein said hydrogel comprises one or more polysaccharides selected from agar, alginate, chitosan, dextran, poly(ethylene glycol), collagen, gelatine, hyaluronic acid, carrageenan, fibroin, fibronectin, poly-l-lysine (PLL), cellulose, graphene, polyethylenimine (PEI), poly(amidoamine) (PAA), dextran sulfate, silk, silk fibroin, pectin, K-carrageenan, Iota carrageenan, gellan gum, guar gum, tragacanth gum, xanthan gum, acacia gum, karaya gum, locust bean gum, or sodium carboxymethyl cellulose (S-CMC).

7. The product according to claim 5, wherein said hydrogel comprises a compound from a group of calcium alginate, gelatin, hyaluronic acid, collagen and chitosan.

8. The product according to claim 5, wherein said gel particles comprises a cross-linked or inter-penetrating alginate network, particularly a calcium cross-linked alginate network.

9. The product according to claim 1, wherein said gel particles comprise micro capsules having a core that is surrounded by a hydrophilic polymer network.

10. The product according to claim 9, wherein said core comprises at least one biologically, pharmaceutically and/or cosmetically active or activatable material from a group comprising: biologicals, anti-oxidants, vitamins, hormones, vaccines, microbiotics, probiotics, prebiotics, antibiotics, enzymes, proteins, fungi, yeast, bacteria, plant cells, mammalian cells and stem cells.

11. The product according to claim 9, wherein said micro-capsules have a fluid core.

12. The product according to claim 9, wherein said micro-capsules have a core that is at least partly solid, particularly having a core that is substantially completely solid.

13. The product according to claim 12, wherein said at least partly solid core comprises a degradable, particularly biodegradable material, specifically a polymer selected form a group, comprising: poly(lactic acid) (PLA), poly(glycolic acid) (PGA), poly(epsilon-caprolactone) (PCL), poly(lactic- co-glycolic acid) (PLGA), as well as modifications of these materials, particularly block copolymers comprising poly(ethylene glycol) (PEG) as one of the blocks, more particularly PEG-PLA, PEG-PLGA, PEG-PGA, or PEG-PCL.

14. The product according to claim 12, wherein said core comprises a mixture of hydrophobic and hydrophilic materials, particularly at least one of said materials being non-homogeneously distributed through said core.

15. The product according to claim 1, wherein at least part of said gel-particles are capable of interacting with biological cells, particularly by being functionalized with one or more compounds selected from a group, comprising: nucleic acids (aptamers), proteins and peptides.

16. The product according to claim 1, wherein said gel particles are biological cell carriers or cell adhesive micro-particles.

17. The product according to claim 1, wherein said gel particles have a size in a range of between 1 micron and 5 millimetre, specifically in a range of between 1 micron and 500 micron.

18. The product according to claim 17, wherein said gel particles contain a pharmaceutically active compound and have a size between 10 and 100 micron and/or said particles contain a nutritious ingredient or supplement and have a size between 100 and 500 micron and/or said gel particles contain a cosmetically active agent and have a size between 500 and 5000 micron.

19. The product according to claim 1, wherein said composition comprises at least 50% of volume of said gel particles and at least 10% of weight of said carbohydrate matrix.

20. The product according to claim 1, wherein said composition is shaped to form at least a portion of a tablet, particularly a tablet that is at least ten times a volume of an average volume of said gel particles.

21. The product according to claim 20, wherein said tablet comprises multiple compartments and said composition is shaped into one of said compartments of said tablet.

22. The product according to claim 1, wherein said shaped slurry is coated by an elastomer compound prior or past solidification, particularly by polydimethylsiloxane (PDMS).

23. The product according to claim 1, wherein said composition moreover comprises an effervescent disintegration agent, particularly a carbonate compound.

24. The product according to claim 1, wherein said composition moreover comprises a plasticizer or other weakening agent, particularly ethanol.

25. A medicinal product comprising a product of the type as claimed in claim 1, wherein said gel particles comprise a pharmaceutically active agent.

26. A beauty product comprising a product of the type as claimed in claim 1, wherein said gel particles comprise a cosmetically active agent.

27. A food product comprising a product of the type as claimed in claim 1, wherein said gel particles comprise a nutritious ingredient or supplement.

28. A method of manufacturing a solid product that comprises a granular composition of gel particles, particularly micro-gel particles, wherein an aqueous slurry is formed comprising said gel particles and a water soluble carbohydrate compound, wherein said slurry is shaped, particularly moulded, and more particularly compressed, into said product, and wherein said shaped slurry, comprising said gel particles and a water soluble carbohydrate compound, is dehydrated and solidified to form said product.

29. The method according to claim 28, wherein said water soluble carbohydrate compound comprises at least one polycarbohydrate or polysaccharide compound.

30. The method according to claim 29, wherein said polycarbohydrate or polysaccharide compound comprises at least one sugar compound.

31. The method according to claim 30, wherein said sugar compound comprises dextran, dextrin, maltodextrin, trehalose, lactose, glucose, dextrose, sucrose, fructose, maltose, isomaltose, sorbitol, mannitol, lactitol, xylitol, and/or erythritol.

32. The method according to claim 28, wherein said shape is solidified by at least substantially removing an aqueous content of said shape, particularly by drying, more particularly by freeze-drying.

33. The method according to claim 32, wherein said slurry is shaped to form at least a portion of a tablet, prior to drying.

34. The method according to claim 33, wherein said tablet comprises multiple compartments and said composition is shaped into one of the compartments of said tablet.

35. The method according to claim 28, wherein said slurry is shaped by moulding, additive material printing or extrusion prior to solidification.

36. A process of forming a slurry that comprises a granular composition of gel particles, particularly micro-gel particles, wherein a solid product according to claim 1 is provided and wherein said product is subjected to a liquid activator.

37. The process according to claim 36, wherein an aqueous liquid activator is used and the product is hydrated.

38. The process according to claim 37, wherein said aqueous liquid activator comprises an aqueous carbohydrate solution.

39. The process according to claim 36, wherein at least one biologically active or activatable material from a group comprising mammalian cells, stem cells, fungi, yeast, bacteria and plant seed is added to said liquid activator.
